(19) United States
(12) Patent Application Publication
(10) Pub. No.: US 2021/0308436 A1
(43) Pub. Date: Oct. 7, 2021

(54) COMPLETE UPPER RESPIRATORY TRACT CLEANSING SYSTEM AND ITS USE

(71) Applicants: James Zhou Liu, Downingtown, PA (US); Yuehue Gu, Kendall Park, NJ (US)

(72) Inventors: James Zhou Liu, Downingtown, PA (US); Yuehue Gu, Kendall Park, NJ (US)

(21) Appl. No.: 14/058,256

(22) Filed: Oct. 20, 2013

(51) Int. Cl. A61M 31/00; A61M 3/02; A61K 33/14; A61K 33/00; A61K 31/194; A61K 36/886; A61K 36/28; A61K 31/352; A61K 31/047; A61K 9/00; A61P 11/02

(52) U.S. Cl. CPC A61M 31/00; A61M 3/0279; A61M 3/0262; A61K 33/14; A61K 33/00; A61K 31/194; A61M 2210/065; A61K 36/28; A61K 31/352; A61K 31/047; A61K 9/007; A61P 11/02; A61M 2210/0618; A61K 36/886

(57) ABSTRACT

The present invention discloses a four-way complete cleansing system for the entire upper respiratory tract (upper airway) and the process of how to use this advanced system to perform a complete cleansing of the upper airway of a human. The device is equipped with two one-way valves and at least two exchangeable tips for an effective upper airway cleansing. The composition of the cleansing solution comprises a chemical compound or an herb extract or the combination having at least one of the cleansing activities against viruses, bacteria, fungi, inflammatory factors, allergens, and promoting membrane healing or immunomodulating, for the treatment and prevention of a respiratory tract disease of a human. The present invention also provides a process for cleansing the hard-to-reach upper respiratory tract, such as sinus, of a mammal to reduce the load of the pathogen, to reduce the amount of inflammatory mediators, to heal pathological changes, and collectively to treat a disease of a mammal, such as nasal allergy or the common cold. This invention also provides a new method of treating a variety of human respiratory tract diseases without adverse effects.

### TECHNICAL FIELD OF THE INVENTION

The present invention relates to a four-way complete cleansing system for the entire upper respiratory tract (upper airway) and the process of how to use this advanced system to perform a complete cleansing of the upper airway of a human. The device is an integral part of the complete cleansing system which is equipped with two one-way valves and at least two exchangeable tips for an effective upper airway cleansing. The composition of the cleansing solution comprises a chemical compound or an herb extract or the combination having at least one of the cleansing activities against viruses, bacteria, fungi, inflammatory factors, allergens, and promoting membrane healing or immunomodulating, for the treatment and prevention of a respiratory tract disease of a human. The present invention also provides a system for delivering the cleansing composition to the hard-to-reach upper respiratory tract, such as sinus, of a mammal to reduce the load of the pathogen, to reduce the amount of inflammatory mediators, to heal pathological changes, and collectively to treat a disease of a mammal, such as nasal allergy or the common cold. The new 4-way cleansing process is provided in detail. This new method of treating a human disease reduces infectious and inflammatory agents, but never causes antibiotic resistance with no adverse event is anticipated.

### BACKGROUND OF THE INVENTION

Respiratory tract viral infections and inflammation caused by allergens are the most common diseases for human, such as nasal allergy and the common cold viruses caused infection. For many of these viral infections, no effective anti-viral drugs are available, therefore, there is a critical need to manage these common viral infections. In the area of respiratory tract bacterial infection, however, as modem antibiotics gradually become less effective due to multiple drug resistance developed by many microbes, termed as superbugs, it becomes harder for pharmaceutical formulation with one or more chemical drugs to achieve therapeutic efficacy for a variety of conditions, such as chronic allergic fungal rhinosinusitis. Hence; an alternative method must be developed to treat these common human respiratory diseases and this present invention is exactly to provide a non-pharmaceutical system to prevent and treat these common respiratory diseases.

According to The American Academy of Otolaryngology—Head and Neck Surgery (AAO-HNS), more than 37 million Americans suffer from at least one episode of sinusitis each year (http://www.entnet.oro/healthinfo/sinus/sinus_questions.cfm). The prevalence of sinusitis has soared in the last decade possibly due to increased pollution, urban sprawl, and the increased number of those microorganisms highly resistant to antibiotics, or super bugs. A recent estimate suggests that 70 million people in America suffer from nasal disorders and a similar number in Europe, making it one of the most common chronic illnesses. However, the treatment of rhinosinusitis is poorly resolved by current drug therapies. The newly invented system here presented with a dual-valve anti-backflow technology is specifically designed to enable the efficient delivery Therapeutic solution by entering into the nose, then into the posterior nasal passage beyond the nasal valve, and finally into sinuses, at the same time allowing the therapeutic solution to fully dissolve infectious and inflammatory materials in nasal and sinus cavities while removing them from those hard to reach areas.

Another example is allergic fungal rhinosinusitis, a hard to treat disease. Mayo study reports showed fungal growth in nasal wash specimens from the sinuses in 96% of patients with chronic sinusitis (Ponikau et al. The diagnosis and incidence of allergic fungal sinusitis. Mayo Clin. Proc. 74:877-884, 1999) while normal controls showed almost the same amount of growth, the difference being that those patients with chronic sinusitis had eosinophils, a type of white blood cell involved in allergic and other reactions, which had become activated. As a result of the activation, the eosinophils release a product called major basic protein (MBP) into the mucus which attacks and kills the fungus but with harsh irritation to the lining of the sinuses and injures the lining of the sinuses which allows the bacteria to proliferate. Furthermore, a number of fungi and bacteria have been detected in sinuses of the patients with chronic rhinosinusitis (Nigro et al. Microbiology of the maxillary and ethmoid sinuses in patients with chronic rhinosinusitis submitted to functional endoscopic sinus surgery. Rev Bras Otorrinolaringol 2006; 72(2):217-222.). Based on the description by Ryan, Matthew, Marple, Bradley (Current Opinion in Otolaryngology & Head & Neck Surgery. 15(1): 18-22, Feb. 2007.), with most patients having detectable fungi, fungal-specific IgE in their allergic mucin while elevated level, of fungal-specific IgG3 being a consistent finding in patients with allergic fungal rhinosinusitis and eosinophilic mucin chronic rhinosinusitis. While antifungal treatment is still considered an option currently, it poses major challenge in reaching an effective blood concentration without causing significant negative side effect. On the other hand, he present invention offers simple solution without anticipated side effect by removing all of above mentioned pathogenic factors: fungi, eosinophils, IgE, IgG3, MBP, and bacteria from nasal and sinus cavities.

Additionally, Influenza viruses A, B and C, including bird flu viruses, respiratory syncytial viruses A and B, SARS virus, parainfluenza viruses and the like are common causes for respiratory tract infections in humans. Presently, due to lack of effective drugs, vaccines or other means to effectively treat or prevent these viral infections prior to identification of the pathogens, human adults and school children are not only constantly exposed to these infectious agents at work and/or at school, but also are possible carriers of these infectious agents to and from their home.

However, with practice of a complete upper airway cleaning, one can effectively reduce the microbial load of those tissues and decrease chance of spreading these disease-causing microorganisms at home, school and working place.

Furthermore, as many disease-causing agents exist in the upper airway, the shedding of communicable infectious microorganisms in the upper airway causes the spread of the disease from the upper respiratory tract to the lower respiratory tract of the carrier. Shedding also causes the spread of these infectious agents to other people through sneezing and/or coughing (Hall, C. B., Douglas, R. J., in an article entitled: “Quantitative shedding patterns of respiratory syncytial virus in infants”,

Journal of Infectious Diseases, 132: 151-156, 1975; and Hall, C. B., Gelman, J. M., Breese, B. B., and Douglas, R. J., in an article entitled “Parainfluenza virus infections in children: Correlation of shedding with clinical manifestations”, Journal of Pediatrics, 91: 194-198, 1977). For most viral infections, antibiotic prevention and/or treatment are generally ineffective. It would therefore be beneficial to have a practical system and/or method to remove these disease-causing agents from the upper airway, in a manner similar to brushing teeth, upper airway delivery of the therapeutic-cleansing liquid effectively and efficiently removes those pathogenic factors from upper airway with no side effect, offering no chance for those microbes to develop drug resistance. By using the new system and process provided in this invention, the general population enjoys number of healthy benefits. Nasal washes, nasopharyngeal swabbing and nasopharyngeal aspiration have been used to obtain specimens from the patients for the determination of microbial pathogens (Hall, C. B., Douglas, R. J., in an article entitled: “Clinically useful method for the isolation of respiratory syntytial virus”, Journal of infectious Diseaces, 131: 1-5, 1975). These procedures and devices were used only to obtain samples and are not effective in removing the infectious agents from the upper airway of a human. Nonetheless, these medical practices indicate that viruses can be removed by using the right kind and amount of the liquid in the current invention. The current invention can resolve this tough issue to meet the unmet need for a human.

Ephedrine nasal washes have been used in the treatment of sinusitis and other nasal and paranasal symptoms and allergic rhinitis, (Shaikh, W. A, in the Journal of Allergy Clinical Immunology, Vol. 96, No. 5, part 1: 597-600, 1995). The Shaikh procedure uses a 1% ephedrine hydrochloride solution in a normal saline solution and a Higginson's rubber syringe. After the rubber syringe is filled with the wash solution, the nozzle of the syringe is introduced into one nostril and the bulb of the syringe is pressed to push the fluid into the nasal cavity. As described by the author, most of the fluid exited from the same nostril, but some fluid exited through the other side of the nose after passing through the nasopharynx. This procedure was performed once every forty-eight (48) hours for a four (4) week period and caused a significant improvement in symptom scores and peak nasal inspiratory flow rates in patients with perennial allergic rhinitis as compared to those treated with a placebo wash (normal saline only). This procedure, however, has the following disadvantages: (1) ephedrine was the key factor for the effectiveness of this procedure, but this chemical is an alkaloid and not suitable for long term daily use by the general public; (2) this procedure may be biased as it was performed only on patients with perennial allergic rhinitis; (3) this procedure was mainly achieving the washing of the nasal cavity only, the sinus and nasopharyngeal cavities were barely cleansed; (4) the apparatus used can re-introduce or re-infect the user by back-flow contaminated solution, 5) clumsy and uncomfortable to use, and can be used incorrectly easily; and (6) this procedure was performed once every forty-eight (48) hours, which is not frequent enough to remove harmful materials from the upper airway on a daily basis. The Shaikh procedure would therefore still allow infectious microorganisms to be brought into and spread around any open space at home, office, school, or day care center. Thus, there is a strong need to develop a generally acceptable, easy to practice, and much more effective upper airway cleaning system and process.

Upper airway is the common place for holding environmental allergens, such as pollen, fungal spores, animal body-originated dusting and volatile chemicals. When inhaled, these harmful agents cause allergic reactions and other ill body reactions and consequences. The secretions from the upper airway combined with environmental particles can further form big matters (solidified mucous) in the nasal cavities. These big matters narrow or block the upper airway and add to nasal obstruction. The presented invention here aims to easily and effectively remove those harmful agents from the upper airway and to prevent the formation of and removal of the big matters in the nasal cavity.

The human body is the natural host for many kinds of pathogenic microorganisms. Sinus, nasopharyngeal and oropharyngeal mucous are prominent places of viral shedding. These pathogens include, but are not limited to, rhinoviruses, influenza viruses, respiratory syncytial viruses and the like. The nasopharyngeal and oropharyngeal shedding of these pathogens is the major cause of person-to-person transmission. It is easy to see the dear advantage and appreciate the significant decrease in quantity of these communicable pathogenic microorganisms present in the nasal, oral, sinus, nasopharyngeal and oropharyngeal cavities after the cavities being largely cleaned. Since after a complete upper airway cleaning, these infectious agents will be less likely to spread horizontally to non-carriers and/or vertically to the lower respiratory tract of the carrier.

Several methods have been reported to be useful in cleaning nasal cavities. Grossan invented a nasal irrigation system (NASAL IRRIGATION SYSTEM, U.S. Pat No. 3,047,145, issued Nov. 12, 1974) which provided for an isotonic saline solution under pressure flowing into one nostril, passing through the nasolacrimal duct, where the solution passes, sequentially, into the ostia of the frontal sinus, the ethnoids, the maxillary and the sphenoid. The solution then moves past the outlet of the

Eustachian tube and then through the nasopharynx to the upper posterior portion of the other nostril and outwardly there Through, passing the same ducts and ostia, in reverse sequence, before being discharged from the second nostril. This system has numerous and serious shortcomings. First, the machine needs electricity, bulky and heavy so it is not convenient for home and traveling use. Secondly, the machine is difficult to clean and has potential water-electric hazard. Thirdly, it is not easy to control the fluid speed by the user. Therefore, the reported nasal irrigation system of Grossan cannot be widely used by the general public for cleaning the upper airway.

A method of administrating a pharmacological solution into the nasal cavity of a patient was described by Lofstedt (US. Pat. No. 5,116,311, issued May 26, 1992). Their device has a compressible container, which not only applies a negative pressure to aspirate and forces the solution flow into the nasal cavity, it easily aspirates or re-infect user with the contaminated solution with nasal secretions and other contaminants back into the container. It is therefore, not an ideal device for nasal drug administration.

Pena invented a device for treating infections of the nasal fossae (U.S. Pat. No. 4,029,095 issued Jun. 14, 1977). The Pena device was also heavy and inconvenient for the patient to administer the drug to treat diseases.

Babbitt et al. invented a portable device used to aspirate and remove fluids from nasal and sinus cavities (U.S. Pat No. 4,403,611 issued Sep. 13, 1983). Since this device only applies a negative pressure to aspirate the sinus fluids from the nasal and sinus cavities, it cannot be used to deliver drug into sinuses.

A variety of neti pots have been commonly used in recent years. However, due to the fact that neti pot can only deliver the solution under gravity, the user must tilt her/his head sideway, the rinse solution can only run from the left to the right nostril, and then from the right to the left nostril. Simply, it is a 2-way cleansing, not a 4-way cleansing. Net pot delivers solution that can only reach and wash nasal passage and some part of the nasopharynx, but not oropharynx or throat. Being the most common places to harbor and incubate viruses and other pathogens, the oropharyngeal cavity and throat remain to be untouched and unwashed, neti pot wash therefore lacks cleansing and drug deliverability related efficiency. The 4-way cleansing process detailed in this invention however demonstrates contrasts its cleansing and drug deliverability efficiency in comparison.

A number of squeeze bottles have been used to rinse the sinuses. However, due to the fad that these squeeze bottles all lack anti-backflow mechanism, the user must remove the bottle from the nostril immediately after squeezing to prevent backflow of contaminated solution which is hard to achieve for most inexperienced user. The manufacturers of these squeeze bottles can only instruct the users to perform the 2-way rinse, the rinse solution can only run from 1) the left to the right nostril, and then 2) from the right to the left nostril. Simply, it is a 2-way cleansing, not a 4-way cleansing. If 2-way rinse is performed, only part of the upper airway can be cleaned. Since these infectious microbes grow to duplicate in the entire upper respiratory tract, not part of upper respiratory tract, an effective prevention and treatment of any respiratory infection needs to dean the entire upper airway. That is the exact goal this current invention is intended to achieve.

Liu, one of the current inventors, presented two inventions related to a nasal-nasopharyngeal-cleaning (NNC) system used to remove harmful substances from a human's nasal and nasopharyngeal cavities (Liu, Nasal-nasopharyngeal-cleaning system, U.S. Pat. No. 6,238,377 May 29, 2001; and U.S. Pat. No. 6,736,792, May 18, 2004). However, the previous two inventions did not teach the 4-way complete cleansing of the upper airway, and the newly invented exchangeable tips for multiple purposes were not included. This current invention is to meet the special need of completely cleaning the entire upper respiratory tract, nostril, nasal passage, sinuses, nasopharynx, oropharynx, throat and mouth in order to be more effective in preventing and treating a human respiratory disease.

### DESCRIPTION OF THE DRAWINGS

That and other objects of the present invention will be apparent to those skilled in the art from the following description of the invention. The invention is described with reference to the following figures:

FIG. 1 is a drawing of the 4-way upper respiratory tract cleansing device. It is a hand-squeezable pump bottle to deliver a liquid into the entire upper airway. The squeezable bottle 1 varies in the liquid holding capacity, from 20 to 500 ml. The liquid is transferred through a thin tube 2 inside of the bottle to the one-directional liquid valve 5 and then is forced to flow to one of the three exchangeable tips, throat wash tip 7, nostril fitting tip 8, oral cleansing tip 9 via the connector 6. The throat wash tip 7 has two ends. The lower end at the label 7 has an out-diameter which fits into the inner diameter of the Connector 6 to form a tight connection. The other end of the throat wash tip at the label 10 is a smooth semi-ball shape with multiple small holes. When the cleansing solution pumped to run out through these many holes, the liquid can rinse and cleanse the throat effectively and still relatively comfortable. The nostril fitting tip 8 can be tightly connected into Connector 6. The other end of the nostril fitting is a semi-ball like with a central hale. The diameter of the central hole can vary from 2 to 6 millimeters. The oral wash tip 9 can be tightly connected to Connector 6. The other end of the oral wash tip 12 is a thin-sharp and smooth structure to allow the liquid flow into the oral cavity, particularly the spaces between the teeth, with strong wash strength. Combinational use of the three exchangeable tips can effectively cleanse the entire upper airway, including nostril, sinuses, nasopharynx, oropharynx, throat, and mouth.

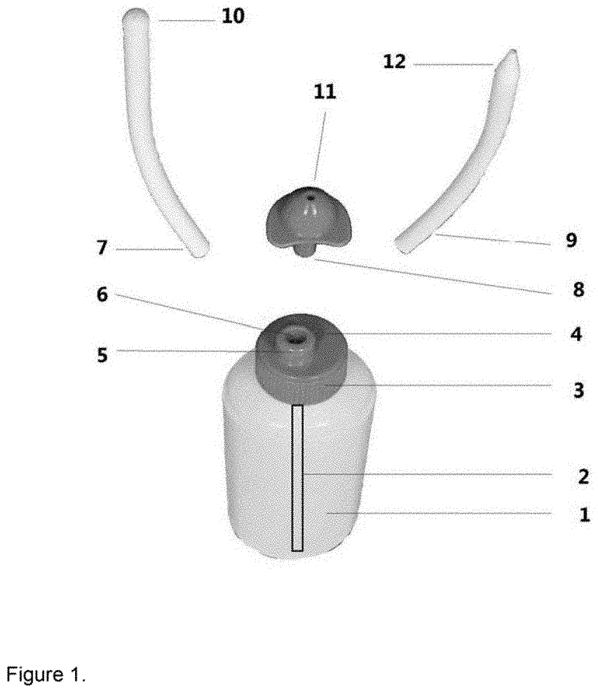

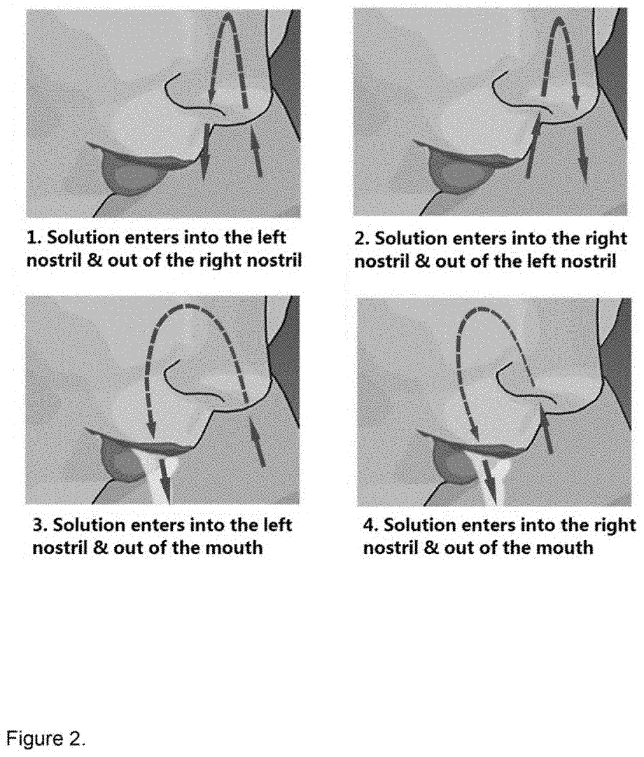

Cap 3 is to seal the bottle to form a pump. Air-valve 4 is to let air enter into the bottle to balance the negative pressure after the bottle is being squeezed then released. When the user releases the bottle after forcing the liquid flow into a human body space, air will enter into the bottle through one-directional air valve 4 while no liquid can flow back into the bottle because the one-way liquid valve 5 stops the liquid back flow. The air valve 5 is to keep the bottle with positive pressure. There will be no negative pressure build up when the liquid is repeatedly forced flow out. Valves 4 and 5 can be made as two parts or can be constructed as one part.

FIG. 2 shows the process of the 4-way upper airway cleansing.

The first way (or Direction 1): the user is to pump the solution to enter into the left nostril, continually flow into the left side of sinuses, run through nasopharynx, finally flow out of the right nostril.

The second way (or Direction 2): the user is to pump the solution to enter into the right nostril, continually flow into the right side of sinuses, run through nasopharynx, finally flow out of the left nostril.

The third way (or Direction 3): the user is to pump the solution to enter into the left nostril, continually flow into left side of sinuses, then the user is instructed to sniff/inhale gently. The continually pump-supplied solution will run through nasopharynx to oropharynx, then run down to throat and end in mouth. The user then spit the wash solution to a sink.

The fourth way (or Direction 4): the user is to pump the solution to enter into the right nostril, continually flow into the right side of sinuses, then the user is instructed to sniff/inhale gently. The continually pump-supplied solution will run through nasopharynx to oropharynx, run down to throat and end in mouth. The user then spit the wash solution to a sink.

To further cleansing throat and oral cavity, the user can switch the throat wash tip and then the oral wash tip to rinse the throat and oral cavities to remove viruses or any harmful materials from the throat and the oral cavity.

It is further disclosed that the 4-way upper airway cleansing system has the following features: 1) the cleansing device is connected with any of the three tips. Any of these tips can be made with different size to be suitable for the different users; 2) said the 4-way upper airway cleansing system is in the dosed position nominally and opens to deliver a solution into the targeted parts of the upper airway upon hand-pumping; 3) said the 4-way upper airway cleansing system has a control means for quantitatively delivering a solution into the specific part of the upper airway; 4) said nostril fitting tip is a one-fit-all object to prevent leakage of the solution from nose openings; 5) said throat wash tip is to spray the solution to the throat and other parts of the oral cavity; 6) said oral tip is to spray the solution into any hard-to-reach space in the oral cavity, such as the space between the teeth; 7) said throat wash tip, nostril fitting tip, and oral wash tip are exchangeable at the Connector 6 on the top of the cap; 8) said throat wash tip is curved or straight to be suitable for any users; 9) said oral wash tip is curved or straight to be suitable for any users; 10) said squeeze bottle is powered or triggered by a human hand; 11) said the 4-way cleansing system disallows liquid back-flow; 12) said 4-way cleansing system can be made with any safe materials; 13) said 4-way cleansing system has a liquid-valve mean to ensure one-directional liquid flow; 14) said 4-way cleansing system has an air-valve mean to maintain a positive pressure within the squeeze bottle;. 15) said 4-way cleansing system can be used for cleaning both upper airway and oral cavity; 16) said 4-way cleansing system is a device to be used to administer a functional liquid to upper airway and oral cavity to reduce the load of pathogenic microbes without causing drug resistance; 17) said 4-way cleansing system is to be used to administer a functional liquid to upper airway and oral cavity to reduce the content of inflammatory agents without disturbing the body's immune function; 18) said 4-way cleansing system is to be used to administer a functional liquid to upper airway and oral cavity in treating respiratory diseases without causing adverse events; 19) said 4-way cleansing system is to be used to administer a functional liquid to upper airway and oral cavity to treat other human diseases without causing the time of a medical professional.

Description of the Composition of the Biocompatible Solution

These and other composition of the present invention will be apparent to those skilled in the art from the following description of the invention. The invention is described with reference to the following composition:

A biocompatible solution for the entire upper airway is very important for the safety and effectiveness of the complete cleansing. A company introduced the wrong rinsing solutions containing detergents at a too high concentration into nasal cavities and caused loss of sense of smell for many users. The solution invented herein is an aqueous solution, an improved normal saline. It is known that the most common isotonic solution for nasal irrigation is a normal saline solution, which contains 9 g of sodium chloride in 1 liter of water (0.9% by weight). This isotonic salt solution, based on the observation obtained from clinical researches, however, can cause dryness of the nasal cavity and it is not optimal to use for a long time. The newly invented solution can contain 9 g of sodium chloride in 1 liter of water, but includes a buffer system and other protective factors. The optimal buffer system is formed by using citric acid and sodium citrate in a correct proportion, such as in a 1:1 ratio to maintain the pH of solution around 7.4. The concentration of sodium chloride can be slightly higher that 9 g/liter, up to 18 g/liter. Sodium bicarbonate has been widely used in a number of nasal irrigation solutions. This ingredient can be used to make a basic formula. The safe ingredient glycerin, a moisturizing agent, is included in the invented formulation. Aloe vera extra, another natural moisturizing agent, is also included. The biocompatible solution may also contain an appropriate amount of an anti-attachment agent, such as carbohydrates which prevent or reduce the attachment of microbial pathogens to the nasopharyngeal and oropharyngeal cavities. The solution may also contain anti-allergy agents, such as eucalyptus oil/eucalyptol. The solution may also contain corticosteroids, antibiotics, antihistamines, and/or mucolytic agents. The solution may also contain a suitable decongestant such as phenylephrine hydrochloride. The temperature of the solution, when administered, is typically about 20 to 40 degree C. and more preferably about 30 to 37 degree C.

Centipeda minima is a herb and has been safely used in the traditional Chinese medicine for the treatment of rhinitis, sinusitis, relieving pain, and reducing nasal swelling. New scientific study shows that the extract of this herb has antioxidant and anti-inflammatory activity, which supports previous observation made during the oral use for relieving inflammation and pain. It is better to use this herb extract locally in the upper airway, not necessarily to let the whole body to have the exposure to this herb. The current invented biocompatible solution is to include the extract from Centipeda minima to treat the upper respiratory tract disorder locally.

Description of the Four-Way Cleansing Process

These and other objects of the present invention will be apparent to those skilled in the art from the following description of the invention.

This invention teaches a new process of the 4-way complete upper airway cleansing. To have a complete upper airway cleansing to remove any harmful substances out of the upper respiratory tract, the user needs to use a hand-pump irrigator with the dual valve anti-backwash function to supply the solution continually. After performs this 4-way cleansing procedure, one can completely cleanse nasal cavity, sinuses, nasopharynx, oropharynx, throat and mouth. Here are the four steps:

Step 1: Pump the cleansing solution run from the left to right nostril. Gently pump the solution into the left nostril, flow pass through nasopharynx, then flow out of the right nostril. One can use about ¼ of the solution (about 60 ml if the bottle holds 240 ml) in the bottle to complete this step. At this time, one can gently blow her/his nose to get rid of the, excessive mucus and leftover liquid. This is mainly to cleanse the left nasal passage, left side sinuses, nasopharynx, and finally, the right side of the nasal passage.

Step 2: Pump the cleansing solution run from the right to the left nostril. Gently pump about 60 ml of solution from the right nostril, let it flow pass nasopharynx, then flow out from the left nostril. One can then gently blow the nose. This is mainly to cleanse the right nasal passage, right side sinuses, nasopharynx from the other direction, and finally, the left side of the nasal passage.

Step 3. Pump the cleansing solution run from the left nostril to throat-mouth. Gently pump about 60 ml of solution into the left side of the nostril. When that side of the nasal cavity is full of the liquid, the user is to gently sniff/inhale the liquid through the nose while continually pumping so the solution will run through oropharynx, throat, and enter into the mouth. Spit the solution to sink. This will cleanse the left nasal passage, left sinuses, nasopharynx, and then the left side of the oropharynx, throat and mouth.

Step 4: Pump the cleansing solution run from the right nostril to throat-mouth. Gently pump the last 60 ml of solution into the right nostril; when the right side nasal cavity is full, gently sniff/inhale the liquid through the nose while continually pumping so the solution runs through oropharynx, throat, and enters the mouth. Spit the solution to a sink. This will cleanse the right nasal passage, right sinuses, nasopharynx, oropharynx, throat and mouth.

After completing the 4-steps of cleansing, one will feel much fresher of the upper respiratory tract. These harmful substances on the surface of the upper respiratory tract will be largely rinse away.

It is not necessary to follow these 4 steps exactly in the order taught herein. Any user can start any one of the 4 steps as the first step or mix any order. When the user has a complete nasal congestion, one can perform Steps 3 and 4 to remove these inflammatory mucus, help drain these sinuses and re-open the nasal cavity. For this purpose, neti pot or these bottles with a functional anti-backflow mechanism will become useless when the nasal cavity is completely blocked.

After performing the above procedure, the user can replace the nostril fitting tip with a throat cleansing tip. Using the remaining liquid in the bottle or freshly made liquid, one can further cleanse the throat area to further remove any pathogens, mucus and inflammatory materials.

Finally, the user can replace the throat cleansing tip with an oral cleansing tip. Using the remaining liquid in the bottle or freshly made liquid, one can further cleanse the mouth to further remove any pathogens, mucus and inflammatory materials.

Description of the Invention of the Method of Use

The present invention further provides a method of treating the common respiratory disorders in a human by effectively removing a variety of pathogens, such as viruses, bacteria, fungi, allergens, environment pollutions, as well as the body produced inflammatory factors, from the upper respiratory tad. After the pathogen is removed from the upper respiratory tract, the body can heal itself. For example, rhinovirus is the cause of the common cold in the upper respiratory tract. The replication of the rhinoviruses triggers the body's immune system to launch an attack against these viruses. The end result of the fight is the inflammation in the upper respiratory tract. When the patient is performing the 4-way complete cleansing procedure to remove these rhinoviruses, the immune system will stop the attack The disease will then disappear.

The present invention further provides a method of preventing common respiratory disorders in a human by effectively removing a variety of pathogens, such as viruses, bacteria, fungi, allergens, environment pollutions, as well as the body produced inflammatory factors, from the upper respiratory tract If the pathogen is removed from the upper respiratory tract before it can cause a disease. Therefore, the disease can be prevented. For example, one individual inhaled infectious aerosols containing the influenza virus during a social gathering. She knows dearly she had a dose contact with a friend who coughed nearby, and she has a high chance to be infected. After leaving that social gathering, she can use the 4-way complete cleansing system to remove any inhaled influenza viruses. Because influenza viruses take time to enter into the epithelium cells to cause infection, timely performing a complete cleansing of the upper respiratory tract, these inhaled influenza viruses will be removed before entering into the cells, hence, the infection can be prevented.

The following examples further illustrate the present invention but, of course, should not be construed as in any way limiting its scope.

### EXAMPLE 1

This example illustrates the efficacy of the 4-way complete cleansing system in treating the common cold. Two groups of the patients with the clinically diagnosed common old were randomly divided into the Treatment or Control groups. In the

Control group, all 8 patients were treated with the currently available medicines for cough and cold. There was no anti-viral drug available. In the Treatment group, these 8 patients were instructed to perform a complete upper airway cleansing, three times a day, using the hand-pump cleansing system. The majority of the patients in the Treatment group recovered 2 or 3 days after starting the upper airway cleansing. The average of duration of the common cold was 3 days. In the Control group, the duration of the cold varied from 7 to 14 days. This dearly demonstrates that the 4-way cleansing procedure with a hand-pump is very effective in treating the common cold.

### EXAMPLE 2

This example illustrates the procedure of performing the complete upper airway cleansing can remove common viruses from the upper respiratory tract. A group of 8 patients were tested to have a positive infection by influenza virus A. They were instructed to perform a complete cleansing of their upper respiratory tract, three times a day, using a hand-pump cleansing system. Three days late, all patients were tested again for influenza viruses. The test result dearly showed that no more viruses could be detected after the three days of performing an effective upper respiratory tract cleansing. These patients recovered within 5 days after the initial infection, which was much faster than the typical influenza virus infection of 10 or more days.

### EXAMPLE 3

This example illustrates the efficacy of using the present invention to remove inflammatory mediators from upper airway cavities of 8 patients with viral infection. The patients with a current viral infection were observed by a physician specialized in Ear, Nose and Throat on Day 1 office visit. All of them had inflammation signs, such as intranasal edema and membrane redness. On Day 4, the same ENT did another observation. Almost all patients had no more inflammation signs. These inflammation signs, such as intranasal edema and membrane redness, were gone. Patients have no more nasal congestion.

### INDUSTRIAL APPLICABILITY

Through the easy use of the 4-way complete upper airway cleansing system, the general population now has a simple, safe, easy and inexpensive method that can be used to remove many kinds of pathogens from the upper respiratory tract to treat and prevent a variety of the common human diseases. This system can also be used to remove inflammatory agents generated by the human body in response to viral infection or reacted towards an allergen. Timely remove infectious particles from the upper respiratory tract will reduce pathogenic microorganism loads and thereby reduce the spread of infection to others, and reduce the opportunity for the disease causing agents to reach his or her lower respiratory tract. The significant advantage of using this new system is that there will be less chance to develop multiple-drug resistance, which is a big public threat in light of many deaths caused by those super bugs. This system also helps the patient to remove harmful inflammation mediators from the upper respiratory tract without using those anti-inflammatory drugs. Hence, those drug's adverse effects and disturbance of immunity can be avoided. The medical community and the general population will greatly benefit from using the new system, and from performing the complete cleansing process and the method disclosed herein. The medical community now has a simple and reliable method to let the patient to remove these pathogens from the upper respiratory tract to treat those disorders.

Those skilled in the art will appreciate that changes and modifications of the upper airway cleansing system can be made, as well as the 4-way cleansing process and the methods of use disclosed herein without departing from the spirit and scope of the present invention as set forth as defined by the following claims.

### REFERENCE

| | | | |
|---|---|---|---|
| 1248891 | Dec., 1917 | Nichols | 604/289. |
| 1487252 | Mar., 1924 | Lore | 604/289. |
| 1502163 | Jul., 1924 | Sprague | 604/217. |
| 2566806 | Sep., 1951 | Miller | 128/173. |
| 2582529 | Jan., 1952 | Curry | 128/200. |
| 2693800 | Nov., 1954 | Caldwell | 604/289. |
| 3847145 | Nov., 1974 | Grossan | 601/160. |
| 4029095 | Jun., 1977 | Pena | 128/250. |
| 4403611 | Sep., 1983 | Babbitt et al. | 604/118. |
| 4980163 | Dec., 1990 | Blackburn et al. | 434/94. |
| 5116311 | May., 1992 | Lofstedt | 604/54. |
| 5183467 | Feb., 1993 | Mouney | 604/140. |
| 5314690 | May., 1994 | Patterson et al. | 424/114. |
| 5505193 | Apr., 1996 | Ballini et al. | 128/200. |
| 5752510 | May., 1998 | Goldstein | 128/200. |
| 5794619 | Aug., 1998 | Edelman et al. | 128/200. |
| 5899878 | May, 1999 | Glassman | 103/849 |
| 6238377 | May, 2001 | Liu | 604/289. |
| 6736792 | May, 2004 | Liu | 598/522 |

### OTHER REFERENCES

A publication entitled. “Quatitative Aspects of Nasal Irrigation Use by Patients With Chronic Sirius Disease in a Multimethod Study”, by David Rabagoin et al., in Ann Fam Med 2006 vol. 4 no. 4, 205-301.

A publication entitled: “Parainfluenza viral infections in children: Correlation of shedding with clinical manifestations” by Hall et al. in The Journal of Pediatrics, 1982, Vol. 91, No. 2, pp. 194-198 .

A publication entitled “Clinically Useful Method for the isolation of Respiratory Syncytial Virus”' by Hall et at in The Journal of infectious Diseases, 1975, Vol. 131, No. 1, pp. 1-5.

A publication entitled=“Quantitative Shedding Patterns a Respiratory Syncytial Virus” in Infants n by Hall et al. in The Journal of Infectious Diseases, 1975, Vol. 132, No. 2, Aug., pp. 151-156.

A publication entitled: Pre- and Postoperative Sinus Penetration of Nasal Grobler et al. in The Laryngosoape, 2008, Volume 118, Issue 11, pages 2078-2081.

A publication entitled: A Novel Method of Drug Delivery for Fibrinolysis with Ho :YAG Laser-Induced Liquid Jet by Hirano et al. in Lasers in Medial Science 2002, 17(3).

A publication entitled=Sounding airflow enhances aerosol delivery into the paranasal sinuses by Maniscalco et al. in European Journal of Clinical Investigation, 2006, Vol. 36, Number 7, July 2006 pp. 509-513(5),

A publication entitled: Drug output from nebulizers is dependent on the method of measurement. P W Barry, C O'Caliaghan. Eur Respir J 1998; Vol. 12: 463-466.

A publication entitled: Saline nasal irrigation for acute upper respiratory tract infections, by Kassel, et al Cochrane Database Syst Rev, May 17, 2010, published online.

A publication entitled: Efficacy of nasal irrigation in the treatment of acute sinusitis in children, by Wang et al. in International journal of Pediatric Otorhinolaryngology, 2009, Volume 73, Issue 12. Pages 1696-1701.

A publication entitled=Baby shampoo nasal irrigations to the symptomatic post-functional endoscopic sinus surgery patient., by Chiu et al., in American journal of Rhinology, 2008, Volume 22, Number 1pp. 34-37(4):

A publication entitled: Impact of pulsatile nasal irrigation on the prognosis of functional endoscopic sinus surgery, by Liang et al. in Journal of Otolaryngology—Head & Neck Surgery 2008, 37(2):148-153.

A publication entitled: Nasal Saline Irrigation Facilitates Control of Allergic Rhinitis by Topical Steroid in Children, by Li et al. in ORL 2009; 71:50-55.

A publication entitled: The diagnosis and incidence of allergic fungal sinusitis, Ponikau, J. U., D. A. Sherris, E. B. Kern, H. A. Homburger, E. Frigas, T. A. Gaffey, and G. D. Roberts. 1999. Mayo Ctn. Proc. 74:877-884.

A publication entitled: The clinical signifince o nasal irrigation bottle contamination, by Keen et al., in The Laiyrigoscope, 2010, Volume issue 10, pages 2110-2114.

A publication entitlect The effect of saline solutions on nasal and mucociliary clearance in rhinosinusilis patients, by Hauptman and Ryan, in Otolaryngology-Head and Neck Surgery, 2007-171.67.121.18.

A publication submitted: Microbiology of the maxillary and ethmoid sinuses in patients with chronic rhinosinusitis submitted to functional endoscopic sinus surgery. By Nigro et al. in Rey Bras Otorrinolaringol 2006, Vol. 72(2): 217-222.

What is claimed is:

1. A complete upper respiratory tact cleansing system comprising:
1) a squeezable-pump irrigator with the anti-backilow function;
2) a sodium chloride based biocompatible liquid for the entire upper respiratory tract;
3) an connector on the cap of the bottle to fit-in at least two exchangeable tips;
4) two or more lips for cleansing the different pails of the upper respiratory tract;
5) a cap for holding the connector for seating the exchangeable hp and for sealing the squeezable bottle;
6) one pair of the air valve and liquid valve in the correct opposite flow directions to maintain continual positive pressure within the squeezable bottle;
7) a tube for transferring liquid from the bottle to the liquid valve.

2. The complete upper respiratory tract cleansing system in accordance with claim 1, wherein the squeezable-pump irrigator holds at least 20 ml of the solution.

3. The complete upper respiratory tract cleansing system in accordance with claim 1, wherein the tips are for cleansing sinonasal cavity, throat and oral cavity of a mammal.

4. The complete upper respiratory tract cleansing system in accordance with claim wherein the connector on the cap of the irrigator is to fit-in the tips for cleansing sinonasal cavity, throat and oral cavity of a mammal.

5. A biocompatible liquid for the entire upper respiratory tract comprising:
1) sodium chloride in water at least 0.6% weight/weight;
2) sodium bicarbonate in water at least 0.001% weight/weight;
3) sodium citrate in water at least 0.00001% weight/weight;
4) citric add in water at least 0.00001% weight/weight;
5) a plant extract in water at least 0.00001% weight/weight;
6) a moisturizing agent in water at least 0.000001% weight/weight;
7) an anti-inflammatory agent in water at least 0.000001% weight/weight.
8) An anti-allengic agent in water at least 0.000001% weight/weight.

6. The biocompatible liquid for the entire upper respiratory tract in accordance with claim 5 wherein the plant extract is Aloe vera extract.

7. The biocompatible liquid for the entire upper respiratory tract, in accordance with claim 5, wherein the moisturizing agent is glycerin.

8. The bicompatible liquid for the entire upper respiratory tract in accordance with claim 5, wherein the anti-inflammation agent is eucalyptol.

9. The biocompatible liquid for the entire upper respiratory tract in accordance with claim 5, wherein the anti-allergic agent is the extract of Centipede minima.

10. The process of using an upper respiratory tract cleansing system comprising:
1) selecting a hand-pump irrigator which can be used to supply the cleansing solution continually;
2) filling the hand-pump irrigator with a biocompatible solution for the entire upper respiratory tract;
3) pumping the solution run from the left nostril to the right nostril and out of the body;
4) pumping the solution run from the right nostril to the left nostril and out of the body;
5) pumping the solution to fulfill the left nasal canal then sniff slightly to let the solution run through the throat and out of the mouth;
6) pumping the solution to fulfill the right nasal canal then sniff slightly to let the solution run through the throat and out of the mouth.

11. The process of using an upper respiratory tract cleansing system in accordance with claim 10, wherein the steps 3) and 4) as specified above can be omitted when the user has a significant nasal congestion The user can directly perform the upper respiratory tract cleansing following steps 5) and 6) as specified in claim 10.

12. A method for reducing a respiratory tract disease comprising:
1) selecting a hand-pump irrigator which can be used to supply the cleansing solution continually;
2) filling the hand-pump irrigator with a biocompatible solution for the entire upper respiratory tract;
3) performing a complete upper respiratory tract cleansing to remove any of the pathogen entered into the upper respiratory tract, and/or to remove an allergic agent, and/or to remove an inflammatory factor generated by the body, and/or to remove an excessive mucus from the upper respiratory tract.

13. The method for reducing a respiratory tract disease in accordance with claim 12, wherein said pathogen is a virus, or a bacterium, or a fungus, or a pollen particle, or an environment pollutant.

14. The method for reducing a respiratory tract disease in accordance with claim 12, wherein said respiratory tract disease is the common cold.

15. The method for reducing a respiratory tract disease in accordance with claim 12, wherein said respiratory tract disease is influenza.

16. The method for reducing a respiratory tract disease in accordance with claim 12, wherein said respiratory tract disease is nasal allergy.

17. The method for reducing a respiratory tract disease in accordance with claim 12, wherein said respiratory tract disease is asthma.

18. The method for reducing a respiratory tract disease in accordance with claim 12, wherein said respiratory tract disease is post nasal drip.

19. The method for reducing a respiratory tract disease in accordance with claim 12, wherein said respiratory tract disease is sinusitis.

20. The method for reducing a respiratory tract disease in accordance with claim 12, wherein said respiratory tract disease is a bacterial infection of the upper respiratory tract, such as sinusitis caused by Staphylococats aureus.
